(19) United States
(12) Patent Application Publication
(10) Pub. No.: US 2004/0102617 A1
(43) Pub. Date: May 27, 2004

(54) Specific acylation of carbohydrate hydroxyls

(76) Inventor: Erich J. Molitor, Midland, MI (US)

Correspondence Address: THE DOW CHEMICAL COMPANY, INTELLECTUAL PROPERTY SECTION, P. O. BOX 1967, MIDLAND, MI, 48641-1967, US

(21) Appl. No.: 10/302,263

(22) Filed: Nov. 21, 2002

(51) Int. Cl. C07H 15/04

(52) U.S. Cl. 536/4.1; 536/116; 536/120

(57) ABSTRACT

The present invention is directed toward a novel process for the preparation of pyranosides protected at the 1, 2, 4, and 6 or the 1, 3, 4, and 6 positions.

### BACKGROUND OF THE INVENTION

1. Field of the Invention

The present invention pertains to the field of carbohydrate chemistry and relates to a novel process for the preparation of protected carbohydrates.

2. Description of the Prior Art

Protected carbohydrates are required for the chemical synthesis of complex carbohydrates, which have significant biomedical importance. This patent describes the selective formation of several protected carbohydrates making use of a key selective benzoylation reaction.

The first compound described is methyl 4,6-O-benzylidene-2-O-(p-chlorobenzoyl)-1-thio-β-D-galactopyranoside (II) prepared by selective benzoylation of methyl 4,6-O-benzylidene-1-thio-β-D-galactopyranoside (I). Compound II is an important intermediate in the construction of several important oligosaccharides. Conventionally, compound II is synthesized in five chemical steps from methyl 1-thio-β-D-galactopyranoside. With the present invention, compound II can be prepared from methyl 1-thio-β-D-galactopyranoside in two chemical steps with a significant increase in the overall yield.

There are no reports of methyl 4,6-O-benzylidene-2-O-(p-chlorobenzoyl)-1-thio-β-D-galactopyranoside in the literature nor are there any reports of a similar compound with a benzoyl group in place of the para-chlorobenzoyl group. The synthesis of a similar compound, ethyl 4,6-benzylidene-2-O-(benzoyl)-1-thio-β-D-galactopyranoside and its regioisomer ethyl 4,6-benzylidene-3-O-(benzoyl)-1-thio-β-D-galactopyranoside was described by Garegg et al.[J. Carbohydr. Chem. 1993, 12(7), 933-954]. The yield of the two compounds was 11 and 74 percent respectively. The compounds were prepared by benzoylation of ethyl 4,6-benzylidene-1-thio-β-D-galactopyranoside with benzoyl chloride in a phase-transfer catalyzed reaction with tetrabutylammonium hydrogen sulphate as the catalyst in dichloromethane and 5% aqueous sodium hydroxide. The major product has the benzoyl group at the 3-position, which is opposite from placing the p-chlorobenzoyl group at the 2-position as described here.

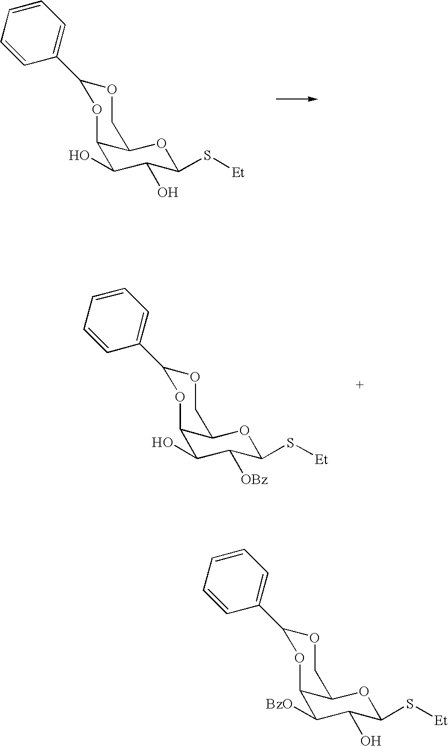

Another closely related example is the benzoylation of n-pentenyl 4,6-O-benzylidene-β-D-galactopyranoside by Udodong et al [Tetrahedron 1992, 48(23), 4713-4724]. Benzoylation with benzoyl chloride under phase-transfer conditions with tetrabutylammonium hydrogen sulphate as the catalyst in methylene chloride and 40% sodium hydroxide gave a mixture of monobenzoylated products that was not separated and in which the ratio was not reported. Nevertheless, a mixture of products was obtained.

The novel process in the present invention produces methyl 4,6-O-benzylidene-2-O-(p-chlorobenzoyl)-1-thio-β-D-galactopyranoside by reacting methyl 4,6-O-benzylidene-1-thio-β-D-galactopyranoside with p-chlorobenzoyl chloride under phase-transfer conditions in 1,2-dichloroethane and 10% aqueous sodium hydroxide. Additionally, the undesired regioisomer (methyl 4,6-O-benzylidene-3-O-(p-chlorobenzoyl)-1-thio-β-D-galactopyranoside)is not produced.

### SUMMARY OF THE INVENTION

The present invention provides a novel process for preparing a compound of the formula

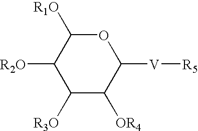

wherein

V is an oxygen or sulphur

R₁ and R₂ represent the hydroxyl protection groups which can be but are not limited to benzyl, benzylidene, tert-butyl diphenyl silyl, tert-butyl dimethyl silyl, trityl, or p-methoxybenzyl.

R₃ is H or an ester protection group

R₄ is H or an ester protection group, and the opposite of R₃

R₅ is C₁ to C₁₀ alkyl, C₁ to C₆ cycloalkyl, aryl, substituted aryl, or carbohydrate

comprising the step of reacting a compound of the formula

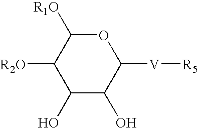

wherein

R₁, R₂, and R₅ are as previously described above is reacted with an acid chloride of the formula

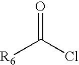

Wherein

R₆ is C₁-C₂₀ alkyl or C₃-C₇ cycloalkyl or an aryl group or a substituted aryl group in a phase transfer catalyzed reaction with, a phase-transfer catalyst of the formula

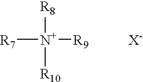

Wherein

R₇ is C₁-C₄ alkyl or benzyl

R₈ is C₁-C₄ alkyl or benzyl

R₉ is C₁-C₄ alkyl or benzyl

R₁₀ is C₁-C₁₆ alkyl or benzyl

X is chloride, bromide, hydroxide, or hydrogensulfate

N is nitrogen or phosphorous

The reaction is carried out in a biphasic system in which the organic phase is toluene, 1,2-dichloroethane, dichloromethane, or other water-insoluble organic solvent and the aqueous phase is a basic solution of sodium hydroxide or potassium hydroxide.

### DETAILED DESCRIPTION OF THE INVENTION

The process of the present invention comprises the selective benzoylation of a carbohydrate such as a galactopyranoside or a glucopyranoside which contains two unprotected secondary alcohol functionalities at the 2 and 3-positions.

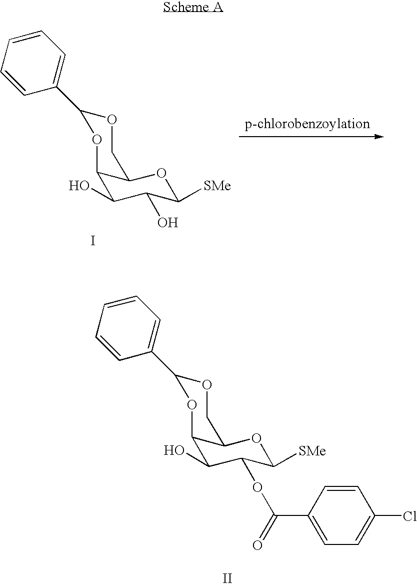

In scheme A, the 2-hydroxyl group of (I) is p-chlorobenzoylated to protected carbohydrate of structure (II).

For example, a solution of (I) and a suitable phase-transfer catalyst such as octyltrimethylammonium bromide in an organic solvent such as 1,2-dichloroethane is cooled to about 0° C. and a solution of aqueous base such as 10% sodium hydroxide is added to form a biphasic system. To this mixture is then added p-chlorobenzoyl chloride. The reaction is quenched by the addition of an aqueous acid solution such as citric acid and the organic phase is separated. The desired product is then isolated by procedures that are well known to one skilled in the art. For example, silica gel chromatography can provide the protected carbohydrate derivative (II).

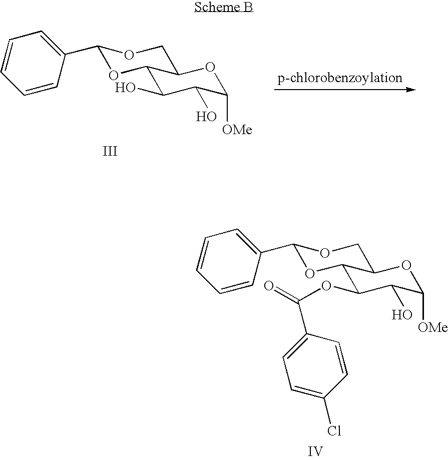

In scheme B, step 1, the 3-hydroxyl group of (III) is p-chlorobenzoylated to produce a protected carbohydrate of structure (IV).

For example, a solution of (III) and a suitable phase-transfer catalyst such as octyltrimethylammonium bromide in an organic solvent such as 1,2-dichloroethane is cooled to about 0° C. and a solution of aqueous base such as 10% sodium hydroxide is added to form a biphasic system. To this mixture is then added p-chlorobenzoyl chloride. The reaction is quenched by the addition of an aqueous acid solution such as citric acid and the organic phase is separated. The desired product is then isolated by procedures that are well known to one skilled in the art. For example, silica gel chromatography can provide the protected carbohydrate derivative (IV).

The following example presents a typical syntheses as described in scheme A. This example is understood to be illustrative only and is not intended to limit the scope of the invention in any way. As used in the following example, the following terms have to the meanings indicated: “g” refers to grams, “mmol” refers to millimoles, “mL” refers to milliliters, “° C.” refers to degress Celsius, and “mg” refers to milligrams.

### EXAMPLE 1

Scheme A: Methyl 4,6-O-benzylidene-1-thio-β-D-galactopyranoside (5.0 g, 16.76 mmol) and n-octyltrimethylammonium bromide (334 mg, 1.32 mmol) were mixed in 1,2-dichloroethane (1,2-DCE, 300 mL). The mixture was thus filtered into a 3-necked 1-L round bottom flask through a paper filter to result in a clear solution. The solution was cooled to 5° C. as paddle stirred and sodium hydroxide (10%, 60 mL) was added. The solution was recooled to 3-4° C., stirred at 310 RPM, and p-chlorobenzoyl chloride (2.1 mL, 16.76 mmol) was added over a period of 4.5 minutes. The reaction was stirred an additional 15.5 minutes with the temperature maintained and was quenched by the addition of 10% citric acid solution (300 mL). The temperature rose to 18° C. and then was recooled to 10° C. The mixture was diluted with water (100 mL) and DCE (65 mL) and the organic phase removed. The aqueous was extracted with additional DCE (95 mL). The combined organics were washed with saturated sodium bicarbonate (100 mL) and water (100 mL) and dried over MgSO₄ and evaporated to give 6.37 g of solid residue. The residue was dissolved in toluene-ethyl acetate (6:1) and loaded onto a pad of silica gel (64 g) and eluted with toluene-ethyl acetate (6:1, 5:1, 4:1, 3:1, and then 2:1). The relevant fractions were combined and evaporated to give 4.67 g (63.8%) of methyl 2-O-(para-chlorobenzoyl)-4,6-O-benzylidene-1-thio-β-D-galactopryanoside as a white solid. Methyl 2,3-di-O-(para-chlorobenzoyl)-4,6-O-benzylidene-1-thio-β-D-galactopyranoside (0.74 g, 7.6%) was also isolated.

What is claimed is:

1. A process for preparing a compound of the formula: 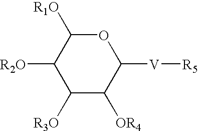
wherein
V is an oxygen or sulphur;
R₁ and R₂, respectively, each represent a hydroxyl protection group selected from the group consisting of benzyl, benzylidene, tert-butyl diphenyl silyl, tert-butyl dimethyl silyl, trityl, and p-methoxybenzyl;
R₃ is H or an ester protection group;
R₄ is H or an ester protection group, and the opposite of R₃;
R₅ is selected from the group consisting of C₁ to C₁₀ alkyl, C₁ to C₆ cycloalkyl, aryl, substituted aryl, and carbohydrate;
wherein the method comprises the step of reacting compounds of the formula 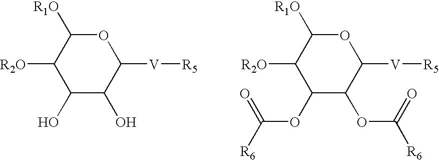
wherein
R₁ and R₂, respectively, each represent a hydroxyl protection group selected from the group consisting of benzyl, benzylidene, tert-butyl diphenyl silyl, tert-butyl dimethyl silyl, trityl, and p-methoxybenzyl;
R₅ is selected from the group consisting of C₁ to C₁₀ alkyl, C₁ to C₆ cycloalkyl, aryl, substituted aryl, and carbohydrate; and
R₆ is selected from the group consisting of C₁-C₂₀ alkyl, C₃-C₇ cycloalkyl, an aryl group and a substituted aryl group
in a phase transfer catalyzed reaction with a phase-transfer catalyst of the formula: 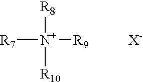
wherein
R₇ is C₁-C₄ alkyl or benzyl;
R₈ is C₁-C₄ alkyl or benzyl;
R₉ is C₁-C₄ alkyl or benzyl;
R₁₀ is C₁-C₁₆ alkyl or benzyl;
X is selected from the group consisting of chloride, bromide, hydroxide, and hydrogensulfate; and
N is nitrogen or phosphorous.

2. The process of claim 1 wherein the reaction is carried out in a biphasic system comprising a liquid organic phase and a basic solid phase.

3. The process of claim 2 wherein the organic phase is a water-insoluble organic solvent.

4. The process of claim 3 wherein the water-insoluble organic solvent is selected from the group consisting of toluene, 1,2-dichloroethane and dichloromethane.

5. The process of claim 2 wherein the solid phase comprises an inorganic base.

6. The process of claim 5 wherein the inorganic base solvent is selected from the group consisting of sodium hydroxide, potassium hydroxide, sodium carbonate and potassium carbonate.
